(19) United States
(12) Patent Application Publication
(10) Pub. No.: US 2016/0045613 A1
(43) Pub. Date: Feb. 18, 2016

(54) PARTICLES HAVING PEG-YLATED SURFACES MODIFIED FOR LYMPHATIC TRAFFICKING

(71) Applicant: THE UNIVERSITY OF NORTH CAROLINA AT CHAPEL HILL, Chapel Hill, NC (US)

(72) Inventors: Joseph DE SIMONE, Chapel Hill, NC (US); Sarah N. MUELLER, Chapel Hill, NC (US)

(21) Appl. No.: 14/782,217

(22) PCT Filed: Apr. 4, 2014
(86) PCT No.: PCT/US2014/032923

§ 371 Date: Oct. 2, 2015

Related U.S. Application Data
(60) Provisional application No. 61/808,898, filed on Apr. 5, 2013.

(51) Int. Cl. A61K 47/48; A61K 39/39; A61K 39/385

(52) U.S. Cl. CPC A61K 47/48215; A61K 39/385; A61K 39/39; A61K 47/48892

(57) ABSTRACT

The subject matter disclosed herein is directed to modifying and utilizing properties of micro and/or nano-particles to traffic the particles to lymph nodes. As described herein, the properties include size, charge, and surface characteristics of the particles.

### CROSS-REFERENCE TO RELATED APPLICATION

This application claims the benefit of U.S. Provisional Patent Application No. 61/808,898, filed Apr. 5, 2013, which is incorporated herein by reference in its entirety.

### GOVERNMENT SUPPORT

This invention was made with government support under Grant No. CA174425 awarded by the National Institutes of Health. The government has certain rights in the invention.

### FIELD OF THE INVENTION

The subject matter herein is directed to micro- and nano-particles exhibiting size, charge and surface properties whereby the particles traffic towards the lymph nodes upon administration.

### BACKGROUND

Vaccines are typically given through a tissue injection such as intra-muscular route; however, the site of action for the vaccine antigens, i.e. antigen presentation to T cells and B cells, and generation of antigen-specific adaptive immunity is the draining lymph nodes (dLN). Dendritic cells are the most professional antigen presentation cells. For efficient antigen presentation, antigens can either be taken up by dendritic cells (DCs) or macrophages and trafficked to the draining lymph nodes, or traffic there themselves to be taken up by LN-resident DCs or interact with B cells directly. Presence of adjuvants may greatly facilitate the activation/maturation of DCs and other cell types to excrete cytokines to recruit other immune cells to the area. The processing of antigens and maturation of DCs allow them to efficiently present the antigen to T cells, which are then able to produce antigen-specific helper T cells and/or killer T cells. Accumulation of antigens in follicular B cell area is required to efficiently crosslink B cells receptors and produce specific antibodies to clear the invading pathogen.

We have previously shown that anionic particles drain much faster and more efficiently than cationic particles. Additionally, the prior art reports that small spherical particles, such as liposomes less than 50 nanometers in diameter traffic more effectively than particles greater than 100 nanometers. However, there is still a need in the art to configure particles for improved trafficking to the lymph nodes for effective vaccine presentation to a patient.

### SUMMARY OF THE INVENTION

The subject matter disclosed herein is directed to modifying and utilizing properties of micro and/or nano-particles to traffic the particles to lymph nodes. As described herein, the properties include size, charge, and surface characteristics of the particles.

In an embodiment, the present invention shows the effects of antigen conjugation and the length of the poly(ethylene glycol) (PEG) linker attaching the antigen (model antigen, ovalbumin (OVA)) to the particle surface.

In certain embodiments of the present invention, surprisingly, the presently described particles having shorter linker length (for example PEG lengths less than about 1000 g/mol) traffic to the lymph nodes more effectively than longer linker length (for example, PEG 5k).

In alternative embodiments of the present invention, surprisingly, the present subject matter describes particles with each dimension greater than 50 nanometers in diameter and that traffic to the lymph node with efficiencies higher than has been shown before.

In further embodiments, surprisingly, the subject matter described herein is directed to particles with a dimension greater than 100 nanometers in diameter and that traffic to the lymph node with efficiencies higher than has been shown before.

In still further embodiments, surprisingly, the subject matter described herein is directed to particles with a dimension less than 100 nanometers in diameter and that traffic to the lymph node with greater efficiency with efficiencies higher than has been shown before.

In still further embodiments, the subject matter described herein is directed to particles with an aspect ratio greater than 1:1 and a largest dimension less than 200 nanometers and that traffic to the lymph node with greater efficiency than particles having a largest dimension greater than 200 nanometers.

### DESCRIPTION OF THE DRAWINGS

FIG. 1 shows a conjugation scheme for attaching the model antigen, OVA, of the present invention to the surface of PRINT particles. It will be appreciated by one of skill in the art that any appropriate antigen may be employed using methods known in the art.

FIG. 2 shows the effects of size and charge on particle (NP) trafficking to the PLN (popliteal lymph nodes) where the particles are not PEGylated or antigen coated. In some embodiments, smaller and negative charged particles traffic to the primary lymph node more readily than larger and/or positive charged particles. According to one embodiment of the present invention 80×180 nm and 80×320 nm negative charged particles show 0.25 to 0.8 times improved trafficking over larger particles and/or positive charged particles of the same composition at 48 hours post injection.

FIG. 3 shows lymph node trafficking of anionic and cationic particles at 2, 24, and 48 hours for multiple size, shape and charge particles according to the present invention. FIG. 3 shows the time-points of 2 and 24 hours from the same experiment of FIG. 2 showing the 48 hour post injection time-point.

FIG. 4 shows the effects of OVA conjugation and PEG linker length on trafficking according to some embodiments of the present invention.

FIGS. 5A & B show time points taken at 5, 15, and 30 minutes for the PEG500OVA particles in addition to the 2, 24, and 48 hour time points done for all other particle sets. Particles were seen in the PLN as early as 5 minutes post injection.

FIG. 6 shows trafficking of hydrogel particles having positive and negative charge with no linker, particles with linker of varying lengths and particles of different sizes.

FIG. 7A-F show, at time point of 2 hours, a high density of NPs are seen in the meduallary area where the T cells are present and in the DC areas as well (particles co-localized with DCs=yellow). (unqunantified, qualitative measure only). This shows that PEGylation helps to facilitate drainage.

FIG. 8A-D show the flow cytometry results when examining the cell populations within the resected PLNs. The blank NPs, PEG₅₀₀OVA NPs, and PEG₅ₖOVA NPs are shown for 80×180 and 1 um particles. The graphs show that the blank NPs are taken up in the highest amount by all four APC cell populations.

FIG. 9A-D show another embodiment of the data from flow cytometry, showing the 48-hour time point.

FIGS. 10A & B show flow cytometry run on resected PLN cells from trafficking experiment in FIGS. 5A&B and 6, respectively.

FIG. 11A-D show the effects of PEG length on immune response comparing PEG₅₀₀ to PEG₅ₖ linker for OVA conjugation onto the hydrogel particle of the present invention.

FIGS. 12A&B show the effects of NP surface groups and how they interact with APCs on immune response.

FIGS. 13A&B depict co-administration of adjuvant on the production of an immune response by 80×180 and 1 um particles.

FIG. 14 shows the 80×180nm NP/SA/OVA particles from the study on FIG. 6, different soluble adjuvants were compared.

FIG. 15 shows the effects of different dosages on the production of an immune response.

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

In embodiments, particles less than 1 micrometer in each dimension provide improved particle trafficking to the popliteal lymph node over larger particles. In other embodiments, particles less than 200 nanometers in each dimension provide improved trafficking to the lymph nodes over larger sized particles. In some embodiments, particles less than 200 nanometers in each dimension and coupled with a PEG linker having a molecular weight of about 500 provide 5 times improved trafficking over particles with PEG linkers having molecular weights greater than 500. In other embodiments, particles less than 100 nanometers in a dimension provide improved trafficking to the lymph nodes over larger sized particles. In some embodiments, particles less than 100 nanometers in a dimension and coupled with a PEG linker having a molecular weight about 500 provide 5 times improved trafficking over particles with PEG linkers having molecular weights greater than 500.

As used herein, the term “particle” or “particles” is intended to mean one or more molded particles. Preferably, the particles comprise a polymer. The particles may further comprise an active agent. Methods of preparing particles are described in US 2011/0182805; US 2009/0028910; US 2009/0061152; WO 2007/024323; US 2009/0220789; US 2007/0264481; US 2010/0028994; US 2010/0196277; WO 2008/106503; US 2010/0151031; WO 2008/100304; WO 2009/041652; PCT/US2010/041797; US 2008/0181958; WO 2009/111588; and WO 2009/132206, each of which is hereby incorporated by reference in their entirety.

The particles are preferably molded wherein the molded particle further comprises a three-dimensional shape substantially mimicking the mold shape and a size less than about 50 micrometers in a broadest dimension. In further embodiments, the particles are preferably molded to have a three-dimensional shape substantially mimicking the mold shape and a size less than about 5 micrometers in a broadest dimension. Preferably, the molded particles have a first dimension of less than about 200 nanometers and a second dimension greater than about 200 nanometers.

According to some embodiments, the composition can further include a plurality of particles, where the particles have a substantially uniform mass, are substantially monodisperse, are substantially monodisperse in size or shape, or are substantially monodisperse in surface area. Within a plurality of substantially monodisperse particles, the amount of PEG-ylation and the molecular weight of the PEG may vary independently or may independently be controlled. In some embodiments, the plurality of particles have a normalized size distribution of between about 0.80 and about 1.20, between about 0.90 and about 1.10, between about 0.95 and about 1.05, between about 0.99 and about 1.01, between about 0.999 and about 1.001. According to some embodiments, the normalized size distribution is selected from the group of a linear size, a volume, a three dimensional shape, surface area, mass, and shape. In yet other embodiments, the plurality of particles includes particles that are monodisperse in surface area, volume, mass, three-dimensional shape, or a broadest linear dimension.

The particles can comprise an active agent. The particles can be formulated into pharmaceutical compositions. In particular, the particles can be formulated into vaccine for administration to a subject. The administration can be accomplished through any route known in the art, e.g., injection or inhalation. These vaccines can further contain any excipient and/or known vaccine components including adjuvants.

The particles described herein are preferably trafficked by the lymphatic system due to the engineered properties of the particles. The lymphatic system is part of the circulatory system and comprises a network of organs, lymph nodes, lymph ducts, and lymph vessels that form and carry a clear fluid called lymph from tissues to the bloodstream. The lymph system is a major part of the body's immune system.

As is known in this field, the term “aspect ratio” refers to the ratio of the longest axis to the shortest axis of a particle. An axis can be any cross-sectional dimension of a particle. In some embodiments, particles can be fabricated with aspect ratios of greater than about 1:1. In some embodiments, particles can be fabricated with aspect rations of greater than about 2:1. In some embodiments, particles can be fabricated with aspect rations of at least about 3:1. In some embodiments, particles can be fabricated with aspect rations of at least about 4:1. In some embodiments, particles can be fabricated with aspect rations of at least about 5:1. In some embodiments, particles can be fabricated with aspect rations of at least about 6:1. In some embodiments, particles can be fabricated with aspect rations of at least about 7:1. In some embodiments, particles can be fabricated with aspect rations of at least about 8:1. In some embodiments, particles can be fabricated with aspect rations of at least about 9:1. In some embodiments, particles can be fabricated with aspect ratios of at least about 10:1. According to some embodiments, particles can be fabricated with aspect ratios ranging from about 1:1 to about 60:1. In alternative embodiments, particles can be fabricated with aspect ratios ranging from about 1:1 to about 50:1. In other embodiments, particles can be fabricated with aspect ratios ranging from about 1:1 to about 40:1. According to some embodiments, particles can be fabricated with aspect ratios ranging from about 1:1 to about 30:1. In yet other embodiments, particles can be fabricated with aspect ratios ranging from about 1:1 to about 20:1. In still further embodiments, particles can be fabricated with aspect ratios ranging from about 1:1 to about 15:1. In still further embodiments, particles can be fabricated with aspect ratios ranging from about 1:1 to about 10:1. In still further embodiments, particles can be fabricated with aspect ratios ranging from about 1:1 to about 9:1. In still further embodiments, particles can be fabricated with aspect ratios ranging from about 1:1 to about 8:1. In still further embodiments, particles can be fabricated with aspect ratios ranging from about 1:1 to about 7:1. In still further embodiments, particles can be fabricated with aspect ratios ranging from about 1:1 to about 6:1. In still further embodiments, particles can be fabricated with aspect ratios ranging from about 1:1 to about 5:1. In still further embodiments, particles can be fabricated with aspect ratios ranging from about 1:1 to about 4:1. In still further embodiments, particles can be fabricated with aspect ratios ranging from about 1:1 to about 3:1. In still further embodiments, particles can be fabricated with aspect ratios ranging from about 1:1 to about 2:1.

The term “PEG” or polyethylene glycol refers to an oligomer or polymer of ethylene oxide. PEG is often described by the molecular weight of the polymer chain. Useful chain lengths are described herein using common terminology.

By “active agent” is intended an agent that may find use in the treatment, diagnosis and/or management of a disease state. Such agents include but are not limited to small molecule pharmaceuticals, therapeutic and diagnostic proteins, immunogenic components, antibodies, DNA and RNA sequences, imaging agents, and other active pharmaceutical ingredients. Exemplary active agents include, without limitation, analgesics, anti-inflammatory agents (including NSAIDs), anticancer agents, antimetabolites, antineoplastic agents, immunosuppressants, antiviral agents, astringents, beta-adrenoceptor blocking agents, blood products and substitutes, contrast media, corticosteroids, diagnostic agents, diagnostic imaging agents, haemostatics, immunological agents, therapeutic proteins, enzymes, lipid regulating agents, prostaglandins, radio-pharmaceuticals, sex hormones (including steroids), anti-allergic agents, stimulants and anoretics, sympathomimetics, xanthines, antibiotics, and antiviral agents.

The term “therapeutically effective amount” as used herein refers to an amount of the plurality of monodisperse particles sufficient to achieve a certain outcome, such as to elicit an immune response in the subject. By “eliciting an immune response” is intended the generation of a specific immune response (or immunogenic response) in a subject. In some embodiments, the immunogenic response is protective or provides protective immunity, in that it enables the vertebrate animal to better resist infection or disease progression from the organism or tumor cell against which the immunogenic composition is directed. The effective amount and dosage of such active agents required to be administered for effective treatment are known in the art or can be readily determined by those of skill in this field. Where active agents do not have a known dosage for certain diseases, the effective amount of active agent and the amount of a particular dosage form required to be administered for effective treatment can be readily determined by those of skill in this field.

By an “effective” amount or a “therapeutically effective amount” of an active agent is also meant a nontoxic but sufficient amount of the agent to provide the desired effect. Of course, the amount of active agent administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, and the like. Determination of an effective dosage is well within the capabilities of those skilled in the art coupled with the general and specific examples disclosed herein.

The amount of active agent present in the pharmaceutical composition will depend on the agent. Most useful agents are indicated for certain diseases and conditions and the dose amount of active agent can be readily determined and a pharmaceutical composition comprising the desired amount can be prepared as disclosed herein. Useful values of active agents are from about 1 mg to about 1,500 mg active agent per dosage form of the pharmaceutical composition. Preferred values are from about 100 mg to about 800 mg. As disclosed elsewhere herein, useful values of active agents include from about 1 μg to about 1,500 μg active agent per dosage form of the pharmaceutical composition. As disclosed elsewhere herein, preferred values are from about 100 μg to about 800 μg.

In an embodiment, the subject matter disclosed herein is directed to a method of treating a subject comprising administering an inventive pharmaceutical formulation as disclosed herein to the subject.

The term “subject” refers to a mammal, which means humans as well as all other warm-blooded mammalian animals. As used herein, the term “mammal” includes a “patient.” As used herein “a mammal in need thereof” may be a subject whom could have been but is not required to have been diagnosed as suffering from the condition intended to be treated.

The term “treating” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease. As used herein the terms “treating” includes “ameliorating,” which refers to all processes wherein there may be a slowing, interrupting, arresting, or stopping of the progression of the condition or symptoms and does not necessarily indicate a total elimination of the underlying condition.

In an embodiment, the subject matter described herein is directed to a micro- or nanoparticle for lymphatic trafficking, comprising:

a polymer particle wherein;
the particle is less than 1 micrometer in each dimension;
the particle has surface coupled PEG polymer chains having an average molecular weight less than about 1000 g/mol.

In a useful aspect of this embodiment, the particle has surface coupled PEG polymer chains having an average molecular weight less than about 500 g/mol. In other useful aspects, the particle has surface coupled PEG polymer chains having an average molecular weight of between about 350 and 650 g/mol. In other useful aspects, the particle has surface coupled PEG polymer chains having an average molecular weight of between about 850 and 1150 g/mol. In other useful aspects, the particle has surface coupled PEG polymer chains having an average molecular weight of between about 400 and 1000 g/mol.

In a useful aspect of these embodiments, the particle/PEG polymer chain complex has a negative zeta potential.

In alternative embodiments, the base hydrogel particles (NPs) were fabricated from the compositions included in the following Table 1:

TABLE 1

| | Cationic | Anionic |
|---|---|---|
| Monomer | Weight Percent | Weight Percent |
| 2-aminoethyl methacylate HCl (AEM) | 10 | 0 |
| PEG₇₀₀ diacrylate (PEGDA) | 20 | 20 |
| Hydroxy PEG₄ acrylate (HP₄A) | 67 | 77 |
| 2,4,6 trimethylbenzoyl | 1 | 1 |
| diphenylphosphine oxide (TPO) | | |
| *DyLight 680 or AlexaFluor 488 | 2 | 2 |
| Total | 100 | 100 |

In some embodiments, the particles of the present invention are PRINT (Liquidia Technologies, Inc., North Carolina) micro- and/or nano-particles.

Table 2 shows the particle characteristics of some embodiments of the particles described herein. The physical dimensions of the particle are reported on the left column, size is measured by dynamic light scattering and reported in nanometers, the polydispersity index, the particles' zeta potential and, if applicable, the OVA loading in μg/mg of particle are all reported. In some embodiments the PEG density on the particles is between about 0.1 and about 0.01 PEG/nm².

TABLE 2

| | | | Zeta | |
|---|---|---|---|---|
| | | | Potential | OVA Loading |
| Size/Shape/Charge | Size (d · nm) | PDI | (mV) | (ug/mg NP) |
| 80 × 180 (−) | 200.8 ± 11.6 | 0.025 ± 0.017 | −24.6 ± 0.3 | — |
| 80 × 180 (+) | 183.8 ± 2.4 | 0.126 ± 0.017 | 42.0 ± 1.5 | — |
| 80 × 320 (−) | 254.7 ± 6.8 | 0.061 ± 0.064 | −31.9 ± 0.2 | — |
| 80 × 320 (+) | 229.0 ± 2.9 | 0.045 ± 0.037 | 44.4 ± 0.3 | — |
| 80 × 2000 (−) | 595.7 ± 2.6 | 0.133 ± 0.051 | −28.7 ± 0.5 | — |
| 80 × 2000 (+) | 370.2 ± 27.7 | 0.069 ± 0.011 | 32.6 ± 1.4 | — |
| 200 × 200 (−) | 251.2 ± 2.1 | 0.128 ± 0.008 | −29.8 ± 0.3 | — |
| 200 × 200 (+) | 256.2 ± 21.8 | 0.104 ± 0.086 | 43.1 ± 1.8 | — |
| 1 um (−) | 1292 ± 189 | — | −30.0 ± 0.8 | — |
| 1 um (+) | 2581 ± 56.6 | — | 22.0 ± 0.7 | — |
| 80 × 180 | 192.0 ± 2.1 | 0.044 ± 0.014 | −39.3 ± 1.6 | 35-90. |
| PEG₅₀₀OVA | | | | |
| 80 × 180 PEG₅ₖOVA | 191.3 ± 0.6 | 0.076 ± 0.004 | −27.5 ± 0.3 | 6.7-102 |
| 1 um PEG₅₀₀OVA | 1459 ± 189.4 | — | −7.0 ± 0.5 | 13-96 |
| 1 um PEG₅ₖOVA | 1238 ± 23.4 | — | −9.6 ± 0.4 | 11-100 |

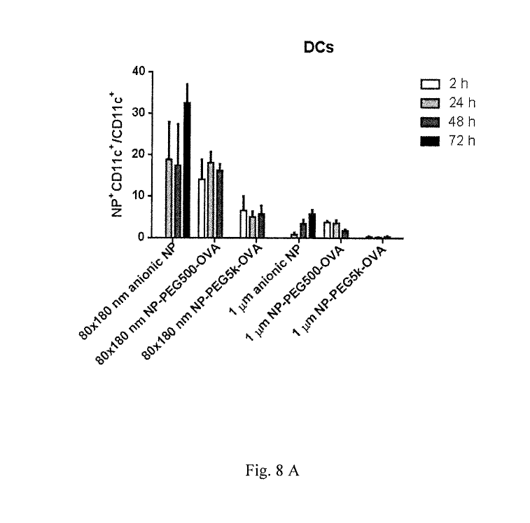

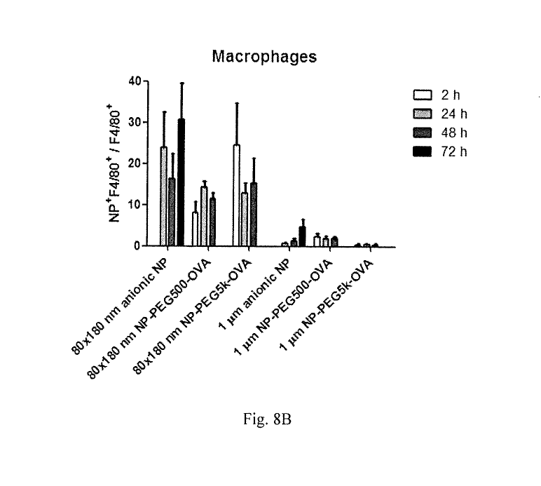

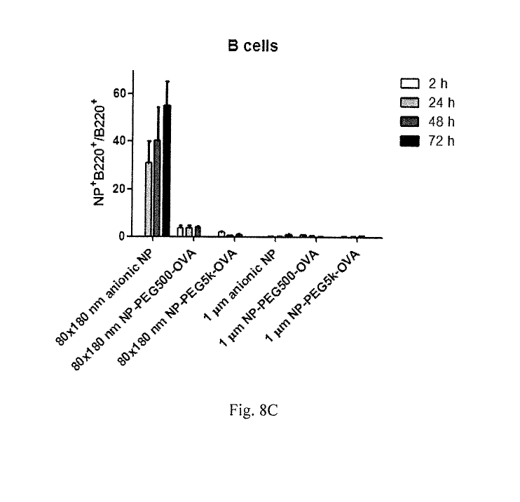

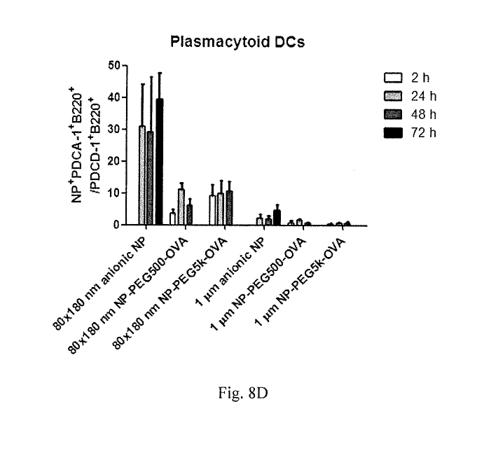

FIG. 8A-D shows flow cytometry results when examining cell populations within resected PLNs. The graphs show that the blank NPs are taken up in the highest amounts by the APC cell populations. However, the 80×180 PEG₅₀₀OVA NPs outperform the 80×180 PEG₅ₖOVA NPs in DC uptake (key to the production of an immune response). The B cell uptake is far higher for the blank NPs as well; however, this non-specific uptake of NPs is not beneficial to eliciting an immune response. Recognition and internalization of antigens (soluble or NP-associated) is important for antigen-specific antibody generation. Non-specific uptake of particles is not helpful unless adjuvants are encapsulated which may activate B cells in antigen-independent way. The B cells must be activated by presentation of the antigen itself, not by uptake of the whole antigen-particle complex.

This data together as a whole shows that there is a trade-off between total trafficking and cell uptake. As disclosed herein, the 80×180 PEG₅₀₀OVA NPs provide particle characteristics and properties that balance the percent trafficked dose and the percent of APCs that take up the NPs.

Described herein are several advantages and unexpected properties of particles having particular PEG-ylated surfaces with designed length and size that provide desirable properties for the particles to be trafficked to the lymphatic system, including the lymph nodes. Surprisingly, in some embodiments, particles having lower chain length PEG are trafficked to the lymph system more efficiently than particles having a higher chain length PEG.

The subject matter disclosed herein includes the following embodiments:

1. A micro- or nanoparticle for lymphatic trafficking, comprising:

a polymer particle wherein;

the particle is less than 1 micrometer in each dimension;

the particle has surface coupled PEG polymer chains having an average molecular weight less than 1000 g/mol; and

the particle/PEG polymer chain complex has a negative zeta potential.

2. The particle of embodiment 1, wherein the PEG polymer chains have a protein coupled with an end not associated with the particle.

3. The particles of embodiments 1 or 2, wherein the particle is less than 500 nanometers in each dimension.

4. The particles of embodiments 1, 2 or 3, wherein the particle is less than 200 nanometers in each dimension.

5. The particles of embodiments 1, 2, 3 or 4, wherein the particles are elongate shaped.

6. The particles of embodiments 1, 2, 3, 4 or 5, wherein the elongate shaped particles have a diameter of less than 100 nanometers across a long-axis of the particle.

7. The particles of embodiments 1, 2, 3, 4, 5 or 6, wherein the particles have an aspect ratio of greater than 1:1.

8. The particles of embodiments 1, 2, 3, 4, 5, 6 or 7, wherein the particle comprises a hydrogel particle.

9. The particles of embodiments 1, 2, 3, 4, 5, 6, 7 or 8, wherein the particle has a positive zeta potential before being coupled with the PEG polymer chains.

10. The particles of embodiment 1, 2, 3, 4, 5, 6, 7, 8 or 9, wherein the PEG polymer chain has an average molecular weight of about 500 g/mol.

11. A method for trafficking active agents in the lymphatic system, comprising:

administering to a patient in need thereof a particle in association with an active agent, wherein the particle comprises; a polymer particle wherein;

the particle is less than 1 micrometer in each dimension;

the particle has surface coupled PEG polymer chains having an average molecular weight of less than 1000 g/mol; and

the particle/PEG polymer chain complex has a negative zeta potential.

12. The method of embodiment 11, wherein the PEG polymer chains have an average molecular weight of about 500 g/mol.

13. The method of embodiment 12, wherein the particle is trafficked toward a lymph node.

14. A method of lymphatic trafficking of an agent, comprising:
administering a plurality of particles to a subject, wherein each particle of the plurality of particles comprises:
   an aspect ratio greater than 1:1;
   a maximum cross-sectional dimension less than 500 nm;
   PEG polymer chains with an average molecular weight less than or equal to 1,000 g/mole coupled with the surface of the particle;
   an agent coupled with the end of a PEG polymer chain not coupled with the surface of the particle; and
   a negative zeta potential in solution.

15. The method of embodiment 14, wherein lymphatic trafficking of each particle of the plurality of particles is greater than lymphatic trafficking of a particle having an aspect ratio of 1:1 or less or a dimension greater than 500 nm.

16. The method of embodiment 14 or 15, wherein each particle of the plurality of particles has a maximum cross-section dimension less than 200 nm.

17. The method of embodiment 14, 15 or 16, wherein each particle of the plurality of particles comprises a polymer.

18. The method of embodiment 14, 15, 16 or 17, wherein the zeta potential is less than −20 mV in solution.

19. The method of embodiment 14, 15, 16, 17 or 18, wherein the aspect ratio is greater than 2:1.

20. The method of embodiment 14, 15, 16, 17, 18 or 19, wherein the maximum dimension of each particle of the plurality of particles is less than 320 nm in cross-section and a smallest dimension of each particle of the plurality of particles is less than 100 nm in cross-section.

21. The method of embodiment 14, 15, 16, 17, 18, 19 or 20, wherein the PEG chains have an average molecular weight of about 500 g/mole.

22. The method of embodiment 14, 15, 16, 17, 18, 19, 20 or 21, wherein the agent is selected from the group consisting of a small molecule drug, a biologic, an antigen, and an adjuvant.

23. The method of embodiment 14, 15, 16, 17, 18, 19, 20, 21 or 22, further comprising a second agent coupled with the end of a PEG polymer chain not coupled with the surface of the particle.

24. The method of embodiment 14, 15, 16, 17, 18, 19, 20, 21, 22 or 22, wherein the lymphatic trafficking is greater than three times the lymphatic trafficking of a particle having a dimension greater than 500 nm or a particle having an aspect ratio of 1:1.

25. The method of embodiment 14, 15, 16, 17, 18, 19, 20, 21, 22, 23 or 24, wherein the polymer particle comprises a biocompatible polymer.

26. A method of delivering an immunogenic agent to a subject, comprising, administering a polymer particle to a patient, wherein the polymer particle comprises; a linker coupled with a surface of the polymer particle; an immunogenic agent coupled with the end of the linker not couple with the surface of the particle; and an aspect ratio greater than 1:1 or each dimension less than 500 nm.

27. The method of embodiment 26, wherein the particle provides enhanced lymphatic trafficking of the immunogenic agent compared to administering a particle having a dimension greater than 500 nm in any dimension or an aspect ratio of 1:1.

28. A particle optimized for delivering an immunogenic agent, comprising:
an aspect ratio greater than 1:1;
a maximum cross-sectional dimension less than 500 nm;
a linker coupled with the surface of the particle;
an immunogenic agent coupled with the end of the linker not coupled with the surface of the particle; and
a negative zeta potential in solution.

29. The particle of embodiment 28, wherein the maximum cross-sectional dimension is less than 320 nm and a smallest cross-sectional dimensional is less than 100 nm.

30. The particle of any one of embodiments 28 and 29, wherein the maximum cross-sectional dimension is less than 200 nm.

31. The particle of any one of embodiments 28, 29 and 30, wherein the zeta potential is less than −20 mV in solution.

32. The particle of any one of embodiments 28, 29, 30 and 31, wherein the particle comprises a biocompatible polymer.

33. The particle of any one of embodiments 28, 29, 30, 31 and 32, wherein the linker comprises a PEG.

34. The particle of any one of embodiments 28, 29, 30, 31, 32 and 33, wherein the linker comprises a PEG having an average molecular weight of less than about 1000 g/mole.

35. The particle of any one of embodiments 28, 29, 30, 31, 32, 33 and 34, wherein the linker comprises a PEG having an average molecular weight of less than about 500 g/mole.

36. The particle of any one of embodiments 28, 29, 30, 31, 32, 33, 34 and 35, wherein the aspect ratio is greater than 2:1.

37. The particle of any one of embodiments 28, 29, 30, 31, 32, 33, 34, 35 and 36, wherein the agent is a small molecule drug, a biologic, an antigen, or an adjuvant.

38. The particle of any one of embodiments 28, 29, 30, 31, 32, 33, 34, 35, 36 and 37, further comprising a second immunogenic agent coupled with the end of the linker not coupled with the surface of the particle.

39. The particle of any one of embodiments 28, 29, 30, 31, 32, 33, 34, 35, 36, 37 and 38, wherein the density of the linker coupled with the surface of the particle is between about 0.1 and about 0.01 linker/nm2.

40. The particle of any any one of embodiments 28, 29, 30, 31, 32, 33, 34, 35, 36, 37, 38 and 39, further comprising an excipient.

41. The particle of any any one of embodiments 28, 29, 30, 31, 32, 33, 34, 35, 36, 37, 38, 39 and 40, wherein the biocompatible polymer is selected from the group consisting of PEG, PLGA, PLA, PGA, and combination thereof.

The present subject matter is further described herein by the following non-limiting examples which further illustrate the invention, and are not intended, nor should they be interpreted to, limit the scope of the invention.

### EXAMPLES

### 1. Effects of Size and Charge on In Vivo Trafficking of Non-PEGylated Particles

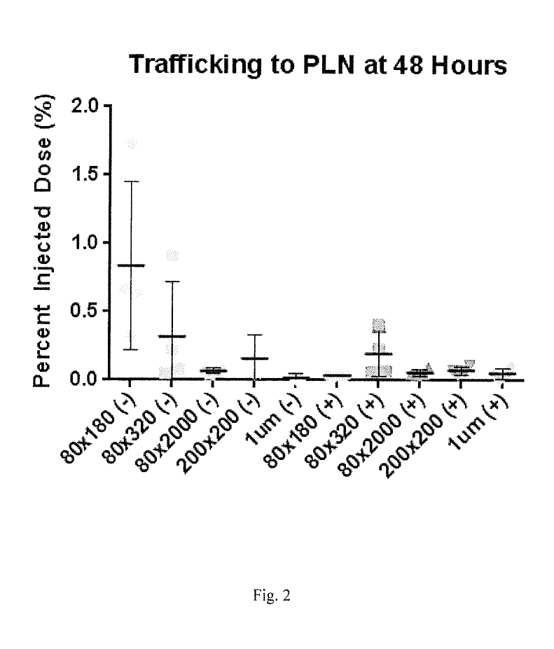

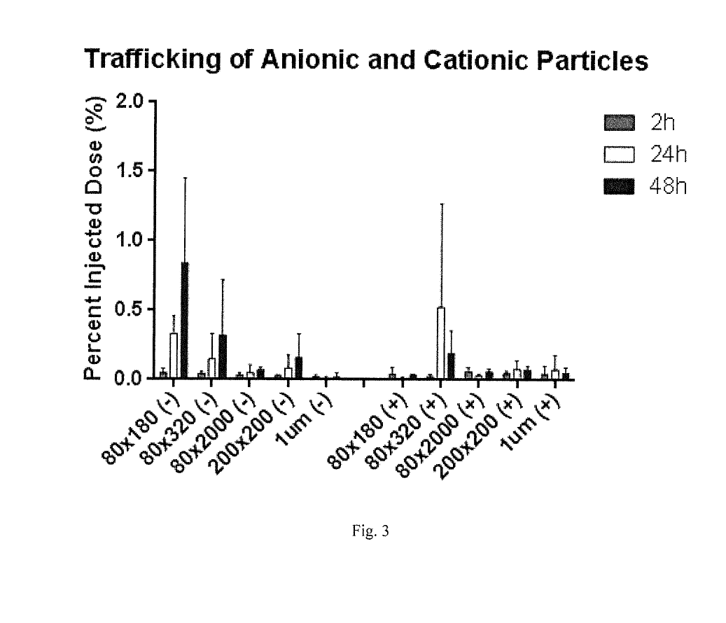

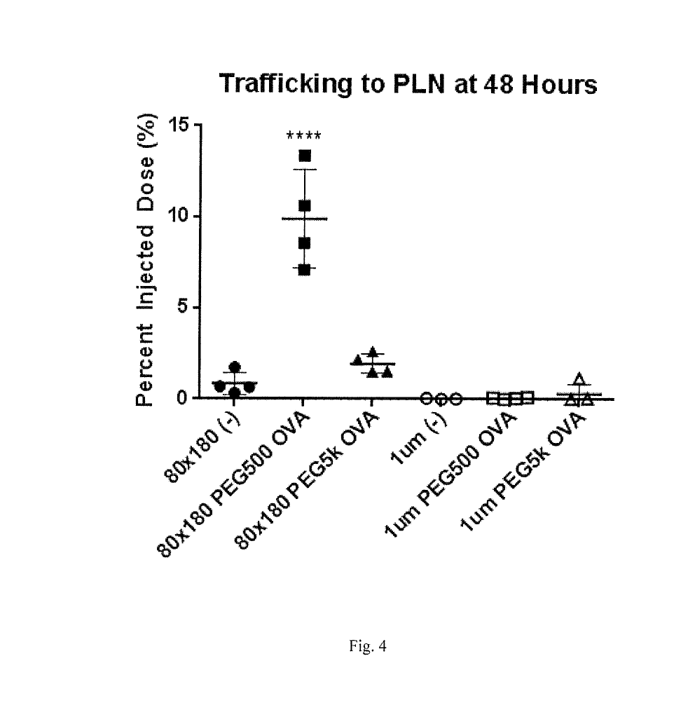

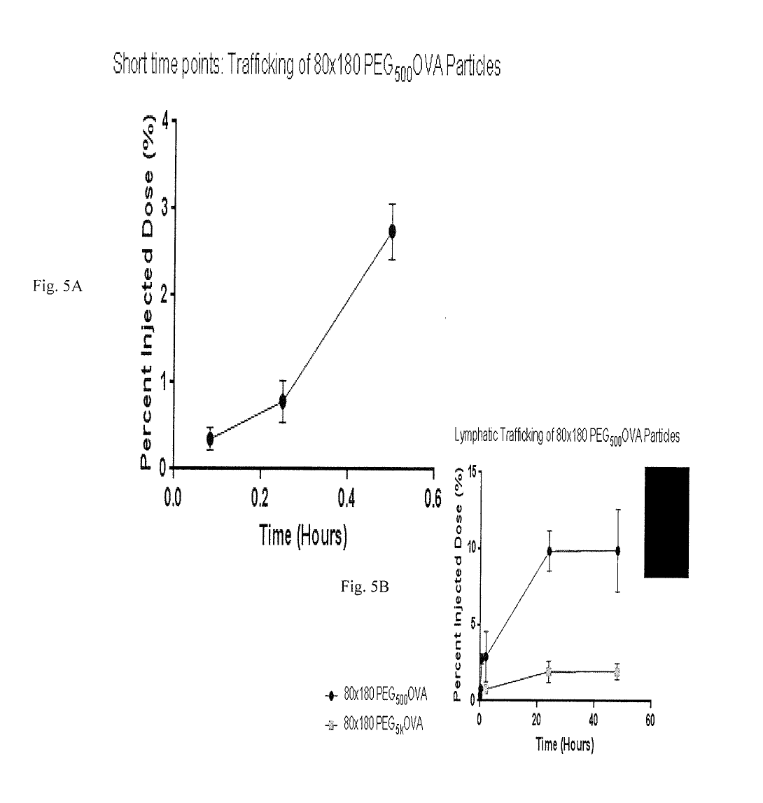

The graph of FIG. 2 shows female balb/c mice, age 6-12 weeks, dosed via footpad injection with 50 μg of fluorescently labeled PRINT particles of various sizes and charges. Mice were sacrificed at 2, 24, and 48 hours post injection. The PLNs, which are the DLNs for this injection site, were resected. The PLNs on the undosed side of the animal were used as the control. Particles showed no drainage past the PLN. The inguinal lymph node and axial lymph nodes were also resected in some cases and showed an absence of NPs. This graph shows that smaller, anionic NPs enhance trafficking to the lymph nodes.

### 2. In Vivo Trafficking of PEG-ylated Particles

The data show down selection to the 80×180 nm rods as a representative small NP and the 1 μm discs as a representative large NP. Each particle size was PEGylated with the bifunctional PEG, with either average molecular weight=500 g/mol or MW=5000 g/mol. OVA was then conjugated to the end of the PEG chains. OVA conjugation was measured through a standard, commercially available BCA assay (Thermo Scientific). Again, mice were dosed with 50 ug of NPs and sacrificed at 2, 24 and 48 hours post injection. Resected PLNs were imaged for fluorescence using an IVIS Lumina instrument in the animal facility at the University of North Carolina, at Chapel Hill. Fluorescence of dosed PLNs was compared to the undosed control PLNs and presented as a percentage trafficked compared to the total dose given. The amount of 80×180 PEG₅₀₀OVA NPs trafficked at 48 hours was 10× greater than the bare 80×180 s and 5× greater than the 80×180 PEG₅₀₀₀OVA NPs. The difference was statistically significant with p=0.0001 (****). FIG. 4 shows the effects of OVA conjugation and PEG linker length.
The draining popliteal lymph nodes and the trans-axial control node were resected at time points 2, 24, and 48 hours post-injection
NP coupled with PEG linker having average molecular weight of less than 1000 g/mol of the present invention traffics faster to the lymph nodes than greater molecular weight PEG linker on the NP. PEG500 linker on a hydrogel polymer core traffics faster to the lymph node than greater molecular weight PEG linker. PEG500 linker on a hydrogel particle traffics 5 to 10 percent more to the lymph node than PEG5k linker and hydrogel particles with no PEG linker on their surface.

FIGS. 5A&B show time points taken at 5, 15, and 30 minutes for the PEG500OVA particles in addition to the 2, 24, and 48 hour time points done for all other particle sets. Particles were seen in the PLN as early as 5 minutes post injection.

### 3. Particle Uptake by DCs, Macrophages and pDCs

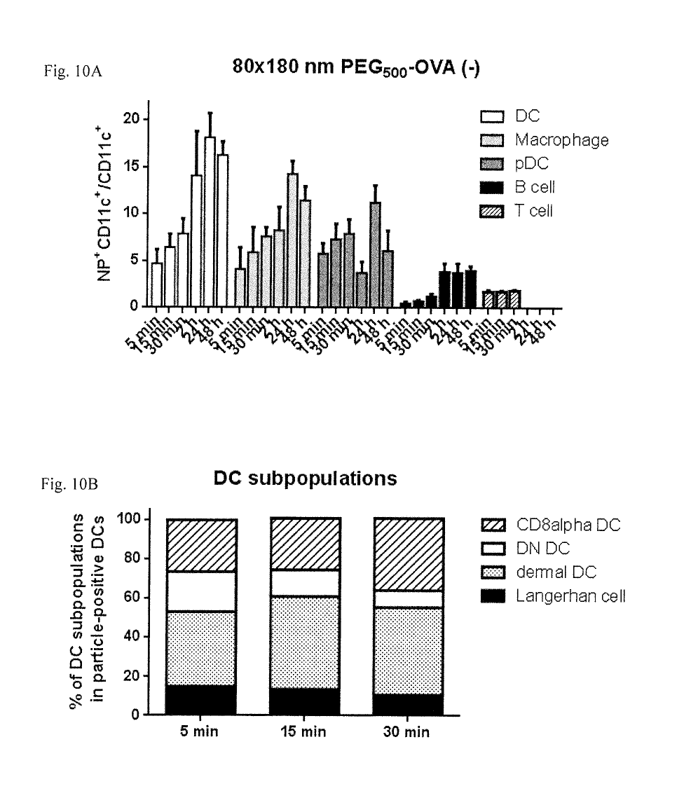

FIGS. 10A&B show flow cytometry on resected PLN cells from the trafficking experiment. In as early as 5 minutes, DCs, macrophages, and pDCs have taken up particles. About 5% of their total populations are positive for particles. Cell mediated trafficking is on the order of 24-hours. Thus, these particles are most likely trafficking through interstitial flow.

Of the DCs that took up NPs, the right graph shows what type of DCs they are: Langerhan and dermal DCs being DCs from the site of injection and draining lymph node (DN) and CD8alpha DCs being LN resident DCs. It was unexpected that dermal DCs were so prevalent in the PLNs, but after doing a subcutaneous injection, this may be caused by the trauma of the needle at injection.

### 4. Elicitation of Immune Response

For the immune response, female balb/c mice age 6-12 weeks were tested. These mice were dosed with 1, 5, or 10 μg of OVA through either a subcutaneous flank injection or a footpad injection. Prime doses were given at day 0, boost doses around day 21. Blood was drawn at day 0, day 21, and day 28. In the cases that adjuvant was administered as well, the adjuvant was co-injected in a soluble form.

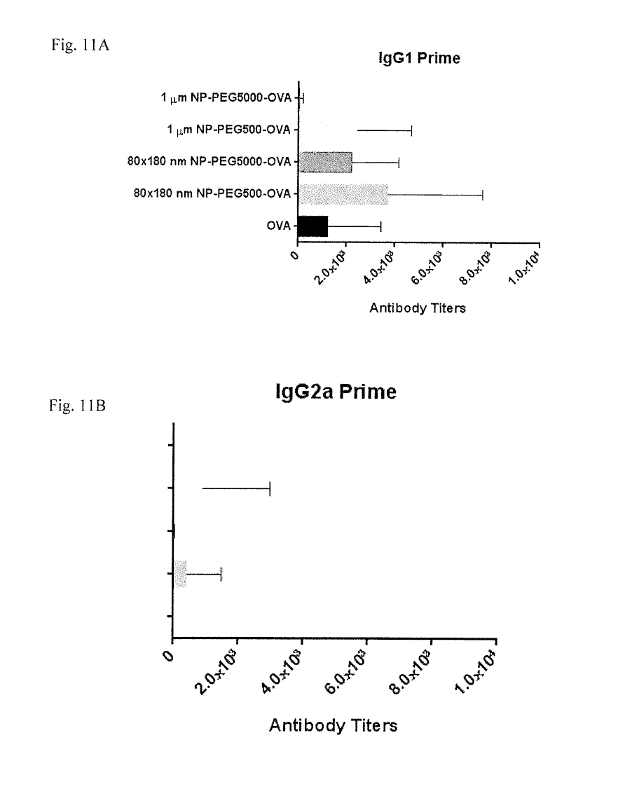

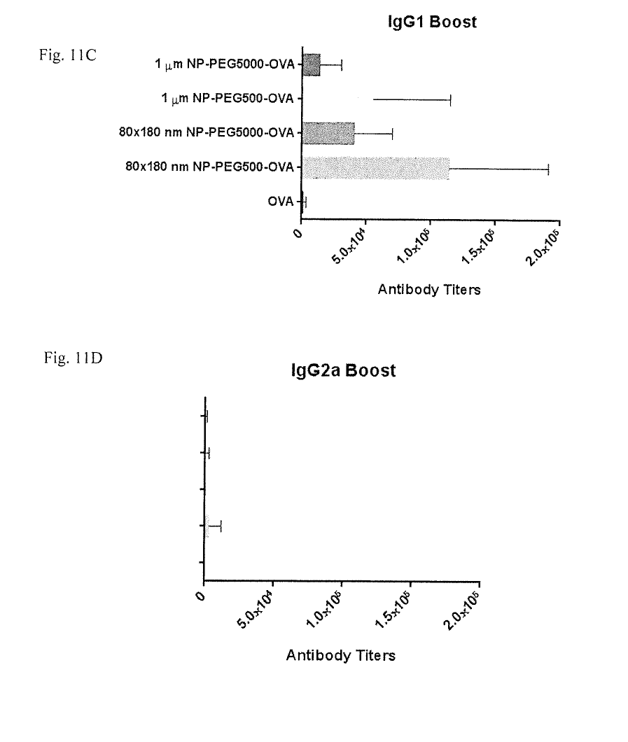

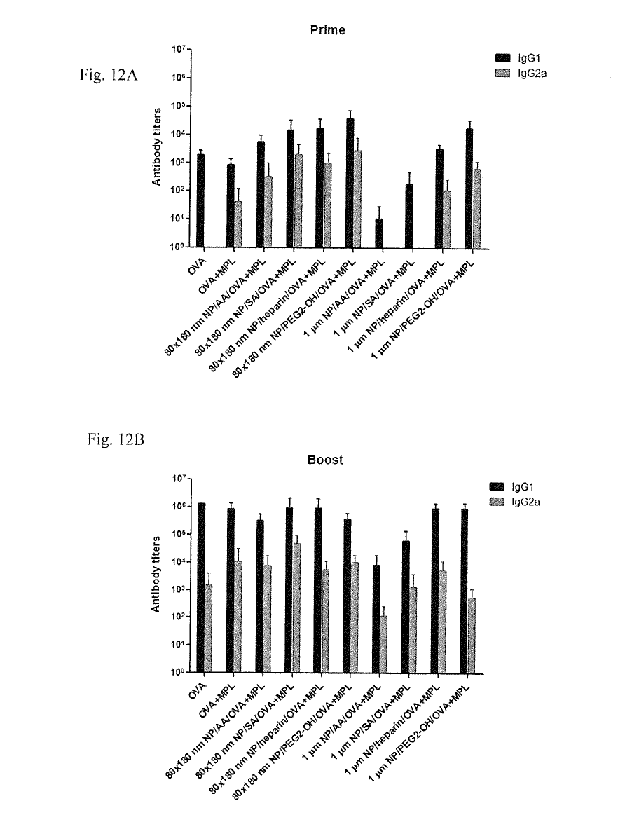

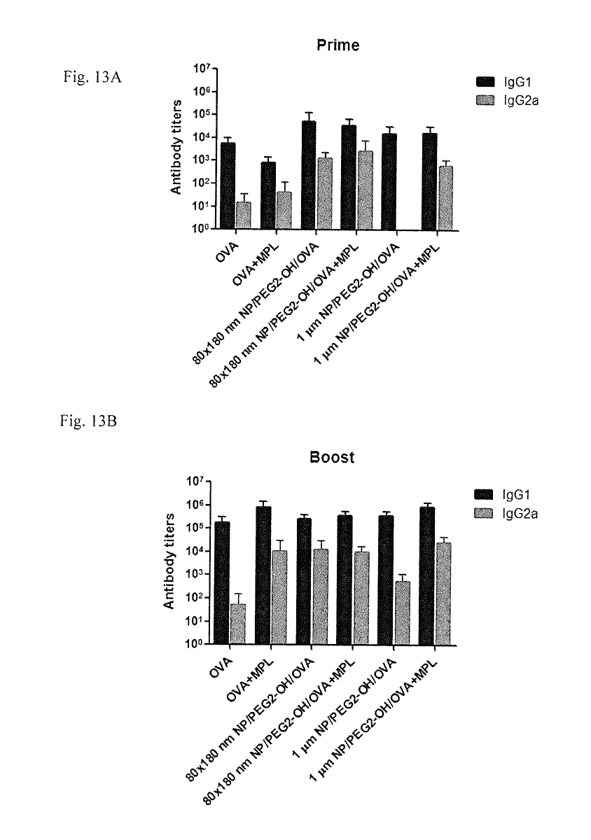

All injections of 5 ug of OVA in 9.25 wt % sucrose were done in the footpad of C57B1/6 mice with an n=4. The prime was given at day zero, the mice were bled at day 21, the boost dose was given at day 23, and the mice were bled again on day 30. Serum was tested for anti-OVA IgG1 and IgG2a by a standard ELISA assay. The graphs of FIG. 11A-D show the immune response elicited by particles with OVA conjugated to the surface by two different PEG linker lengths. After only the prime dose, there is a clear benefit to the particle-mediated delivery of OVA in IgG1 response with both PEG lengths on the 80×180 particles and the 1 um PEG₅₀₀ particles. The IgG2a response is higher for both sized particles with the PEG₅₀₀ linker. After the boost dose, the IgG1 response elicited by the 80×180 PEG₅₀₀ particles is statistically significant compared to the soluble OVA (**p<0.01). The IgG2a response is higher for the PEG₅₀₀ particles of both sizes as well, but is a lower magnitude than that IgG1 response. The 80×180 PEG₅₀₀ particles offer a benefit in efficacy of OVA immune response over the soluble delivery of OVA.

### 5. Effect of NP Surface Groups on Immune Response

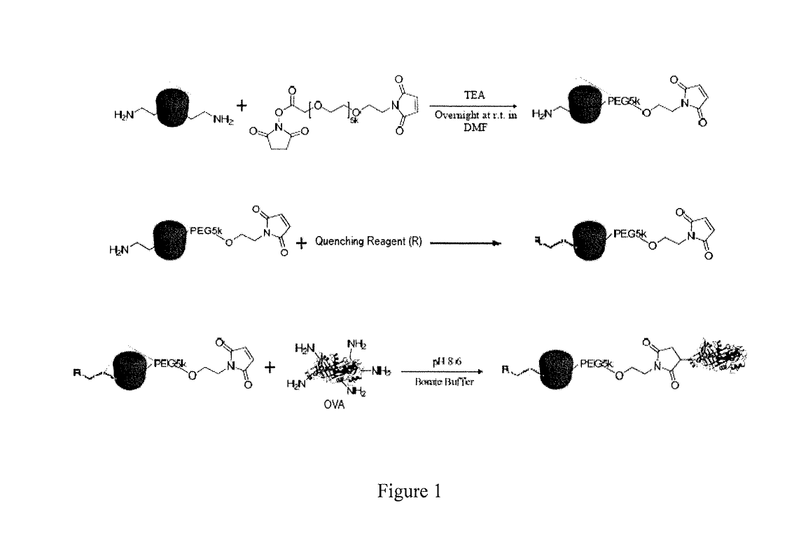

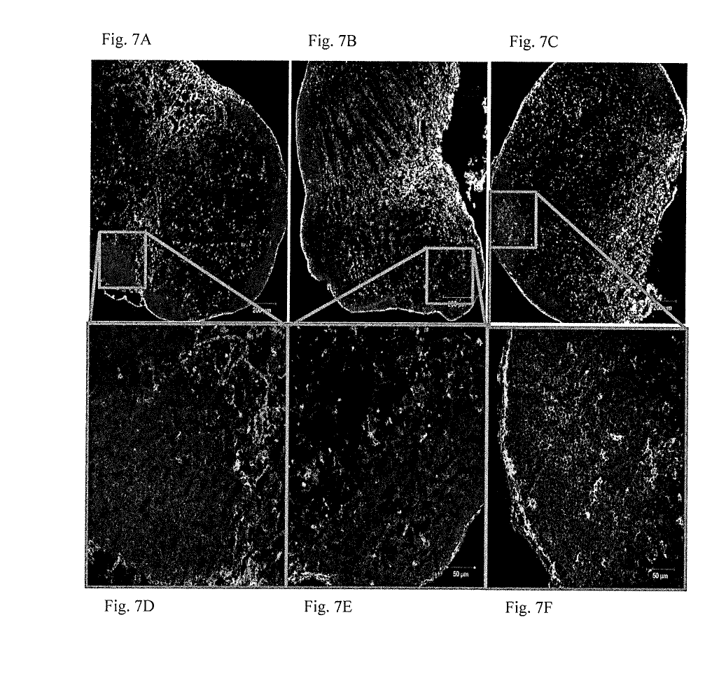

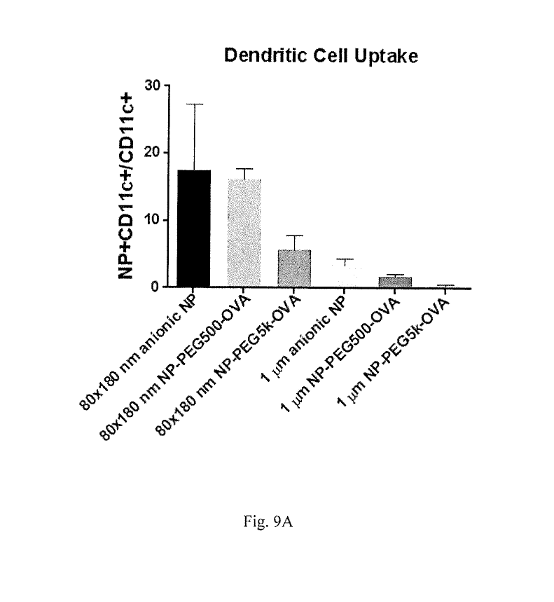

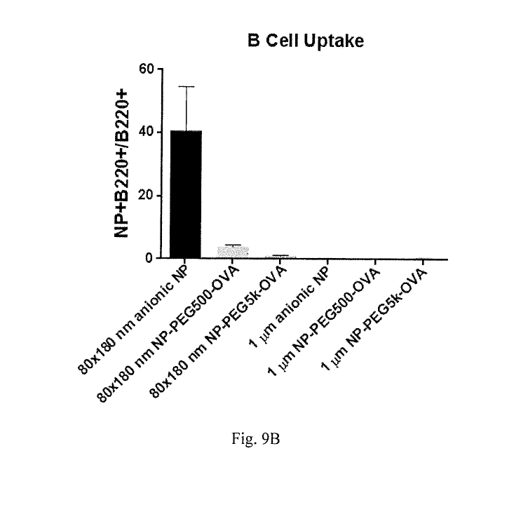

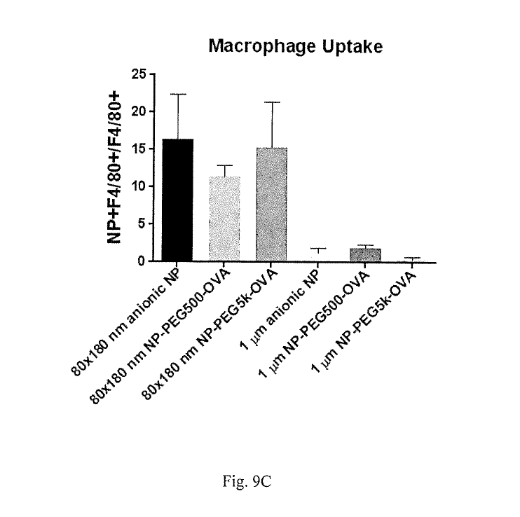

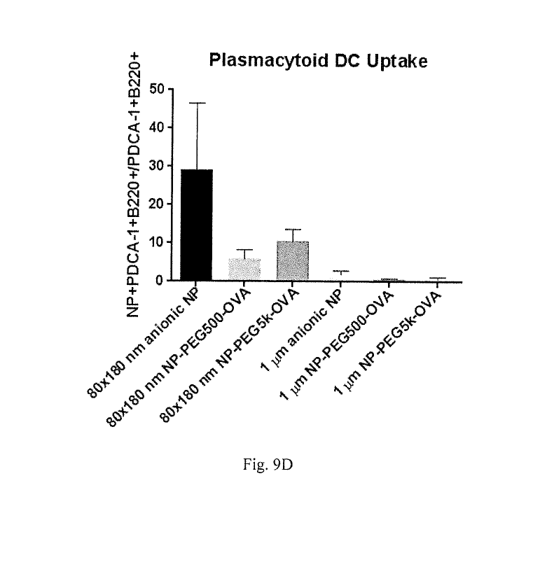

In this study, NPs were quenched (see FIG. 1) with various reagents: AA=acetic anhydride, SA=succinic anhydride, heparin, or NHS-PEG₂OH (quenching method used for NPs in all data for FIGS. 4-12). The data shows the effects of the surface groups on the immune response produced. The 80×180 s show only small differences among the different quenching reagents, while the 1 um particles show higher impact from the quenching reagent used. After the boost dose, the trends are less dramatic, but are still present. After the prime dose, all 80×180 NP formulations outperformed the soluble OVA, with and without adjuvant; however, after the boost dose, they show the same magnitude of response as the soluble administration. This is typical of the balb/c mouse response to OVA. Balb/c mice are Th2-biased model. Thus high IgG1 (Th2 response) production to OVA, an allergen, is not surprising. C57BL/6 is a better model because 1) it's not Th2 biased; 2) it contains the matching MHC class I (Kb) for the major dominating OVA CD8 T epitope SIINFEKL, thus CTL response can be observed under certain conditions. Current studies are being done with a C57B/6 mouse model, noted in literature to be a more suitable model for OVA antigen to test both antibody and T cell responses. The data in FIGS. 12A&B depict the effects of NP surface groups and their interactions with APCs.

### 6. Effect of Co-Administration of Adjuvant

This study, done concurrently with the experiment in Example 5 and the data shown in FIGS. 12A&B, shows the effects of adjuvant administration with the PEG₅ₖOVA NPs, quenched with PEG₂OH. Data from NP/PEG2−OH/OVA+MPL is compared to the same particles administered without adjuvant (NP/PEG2−OH/OVA). Compared to soluble OVA, 80×180 nm particle-mediated delivery of OVA improves IgG1 level by 10 fold, and IgG2a by 50 fold after priming dose. After boost dose, 80×180 nm NP-OVA increased IgG1 by 2000 fold, and IgG2a by 200 fold. The adjuvanticity of 80×180 nm NPs alone without MPL is almost comparable to MPL. The 80×180 particles show the similar response with and without adjuvant while the 1 um particles are more dependent on the soluble adjuvant for a more balanced immune response (class switching from IgG1 to IgG2a).

### 7. Effects of Different Adjuvants on Immune Response

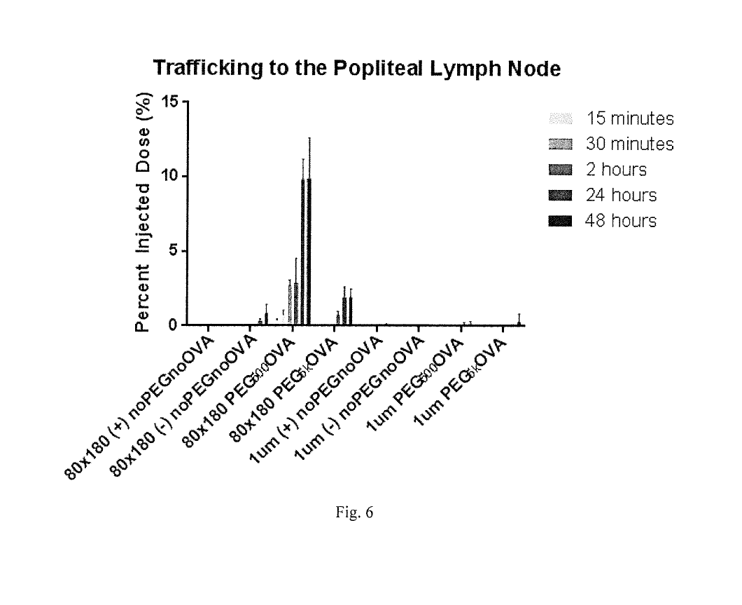

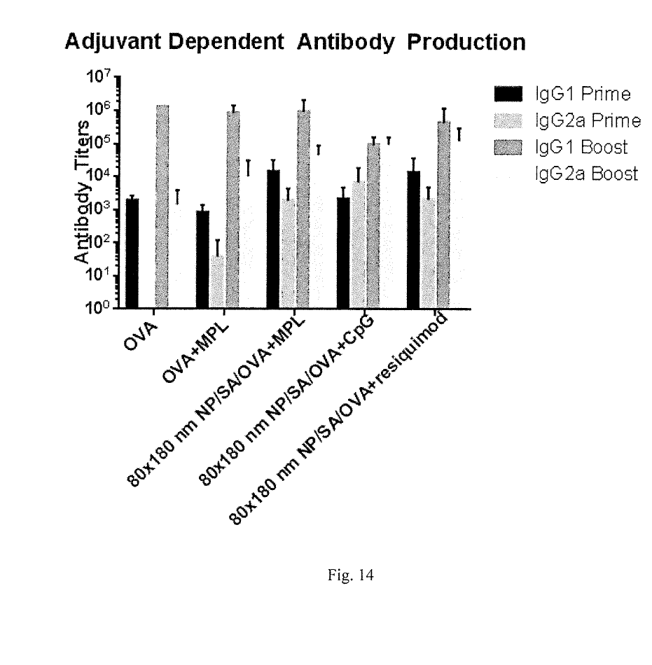

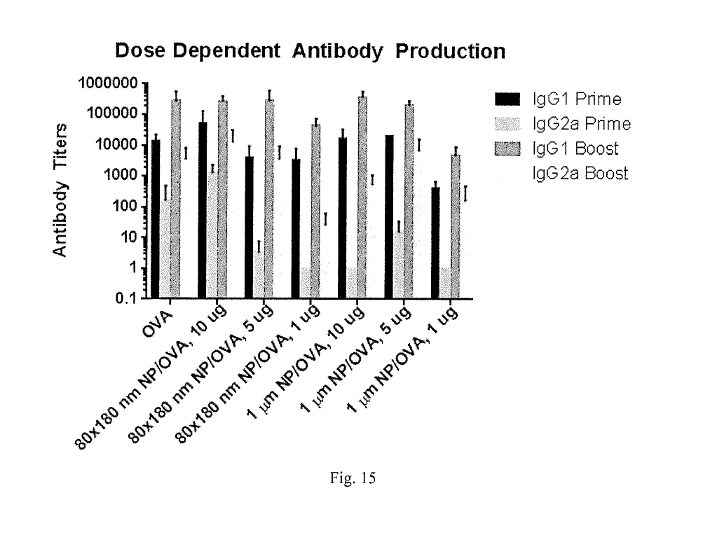

A study on the types of particles used to generate the data in FIG. 6 was performed to identify the effects of various soluble adjuvants on the elicited immune response. MPL activates an extracellular toll-like receptor 4 (TLR4) while CpG and resiquimod activate intracellular TLRs 9 and 7. The data in FIG. 14 show that the particles with all three soluble adjuvants elicit comparable immune responses.

### 8. Effects of Different Dosages on Immune Response

A comparison of 80×180 nm NP/OVA/PEG2OH particles (10 μg dose from study on slide 10) across three dosages was performed. The graph shows the dose dependence of the immune response. For the 10 μg dose, mice were injected subcutaneously in the flank while the 5 μg and 1 μg dose were administered via footpad injection. For the 80×180 s, the immune response after the boots dose is still robust with a 5 μg dose while the lug dose response is significantly lower. For the 1 μm particles, the 10 μg and 5 μg dose are similar as well. The magnitude of the IgG1 response is higher for the 10 μg dose, but the response is more balanced for the 5 μg dose. Again, the 1 μg dose response is significantly lower than the higher doses (50-100 fold in IgG1).

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

That which is claimed:

1. A method of lymphatic trafficking of an agent, comprising:
administering a plurality of particles to a subject, wherein each particle of the plurality of particles comprises:
an aspect ratio greater than 1:1;
a maximum cross-sectional dimension less than 500 nm;
PEG polymer chains with an average molecular weight less than or equal to 1,000 g/mole coupled with the surface of the particle;
an agent coupled with the end of a PEG polymer chain not coupled with the surface of the particle; and
a negative zeta potential in solution.

2. The method of claim 1, wherein lymphatic trafficking of each particle of the plurality of particles is greater than lymphatic trafficking of a particle having an aspect ratio of 1:1 or less or a dimension greater than 500 nm.

3. The method of claim 1, wherein each particle of the plurality of particles has a maximum cross-sectional dimension less than 200 nm.

4. The method of claim 1, wherein each particle of the plurality of particles comprises a polymer.

5. The method of claim 1, wherein the zeta potential is less than −20 mV in solution.

6. The method of claim 2, wherein the aspect ratio is greater than 2:1.

7. The method of claim 1, wherein the maximum dimension of each particle of the plurality of particles is less than about 320 nm in cross-section and a smallest dimension of each particle of the plurality of particles is less than 100 nm in cross-section.

8. The method of claim 1, wherein the PEG chains have an average molecular weight of about 500 g/mole.

9. The method of claim 1, wherein the agent is selected from the group consisting of a small molecule drug, a biologic, an antigen, and an adjuvant.

10. The method of claim 1, further comprising a second agent coupled with the end of a PEG polymer chain not coupled with the surface of the particle.

11. The method of claim 2, wherein the lymphatic trafficking is greater than three times the lymphatic trafficking of a particle having a dimension greater than 500 nm or a particle having an aspect ratio of 1:1.

12. The method of claim 11, wherein the polymer particle comprises a biocompatible polymer.

13. A method of delivering an immunogenic agent to a subject, comprising:
administering a polymer particle to a patient, wherein the polymer particle comprises;
a linker coupled with a surface of the polymer particle;
an immunogenic agent coupled with the end of the linker not couple with the surface of the particle; and
an aspect ratio greater than 1:1 or each dimension less than 500 nm.

14. The method of claim 13, wherein the particle provides enhanced lymphatic trafficking of the immunogenic agent compared to administering a particle having a dimension greater than 500 nm in any dimension or an aspect ratio of 1:1.

15. A particle optimized for delivering an immunogenic agent, comprising:
an aspect ratio greater than 1:1;
a maximum cross-sectional dimension less than 500 nm;
a linker coupled with the surface of the particle;
an immunogenic agent coupled with the end of the linker not coupled with the surface of the particle; and
a negative zeta potential in solution.

16. The particle of claim 15, wherein the maximum cross-sectional dimension is less than about 320 nm and a smallest cross-sectional dimension is less than 100 nm.

17. The particle of any one of claims 15 and 16, wherein the maximum cross-sectional dimension is less than 200 nm.

18. The particle of any one of claims 15, 16 and 17, wherein the zeta potential is less than −20 mV in solution.

19. The particle of any one of claims 15, 16, 17 and 18, wherein the particle comprises a biocompatible polymer.

20. The particle of any one of claims 15, 16, 17, 18 and 19, wherein the linker comprises a PEG.

21. The particle of any one of claims 15, 16, 17, 18, 19 and 20, wherein the linker comprises a PEG having an average molecular weight of less than about 1000 g/mole.

22. The particle of any one of claims 15, 16, 17, 18, 19, 20 and 21, wherein the linker comprises a PEG having an average molecular weight of less than about 500 g/mole.

23. The particle of any one of claims 15, 16, 17, 18, 19, 20, 21 and 22, wherein the aspect ratio is greater than 2:1.

24. The particle of any one of claims 15, 16, 17, 18, 19, 20, 21, 22 and 23, wherein the agent is selected from the group consisting of a small molecule drug, a biologic, an antigen, and an adjuvant.

25. The particle of any one of claims 15, 16, 17, 18, 19, 20, 21, 22, 23 and 24, further comprising a second immunogenic agent coupled with the end of the linker not coupled with the surface of the particle.

26. The particle of any one of claims 15, 16, 17, 18, 19, 20, 21, 22, 23, 24 and 25, wherein the density of the linker coupled with the surface of the particle is between about 0.1 and about 0.01 linker/nm².

27. The particle of any any one of claims 15, 16, 17, 18, 19, 20, 21, 22, 23, 24, 25 and 26, further comprising an excipient.

28. The particle of any any one of claims 15, 16, 17, 18, 19, 20, 21, 22, 23, 24, 25, 26 and 27, wherein the biocompatible polymer is selected from the group consisting of PEG, PLGA, PLA, PGA, and combinations thereof.
